(19) United States
(12) Patent Application Publication
(10) Pub. No.: US 2020/0239820 A1
(43) Pub. Date: Jul. 30, 2020

(54) REACTOR WITH BUILT-IN LIGHTING AND HEATING DEVICE

(71) Applicant: FERMENTALG, Libourne (FR)

(72) Inventor: Olivier CAGNAC, Libourne (FR)

(21) Appl. No.: 16/639,644

(22) PCT Filed: Aug. 17, 2018
(86) PCT No.: PCT/EP2018/072371

§ 371 Date: Feb. 17, 2020

(30) Foreign Application Priority Data
Aug. 18, 2017 (FR) 1757745

(51) Int. Cl. C12M 1/00; C12M 1/02

(52) U.S. Cl. CPC C12M 21/02; C12M 41/22; C12M 31/10

(57) ABSTRACT

The present invention relates to a reactor including a tank (81) intended to contain a mass to be treated and at least one lighting and heating device (83) intended to promote the treatment of this mass, remarkable in that the lighting and heating device comprises:
a plate including at least one groove extending longitudinally and composed of a bottom and two side walls,
illumination means in thermal contact with the bottom of the groove, so that the heat generated by the illumination means is transmitted to the mass to be treated via the bottom of the plate.

### FIELD OF THE INVENTION

The present invention relates to the general technical field of reactors with integrated lighting, in particular for the culture of photosensitive microorganisms.

It can be a bioreactor but also a chemical or physicochemical reactor

### BACKGROUND OF THE INVENTION

The concept of bioreactor, or biological reactor, refers here to a reactor within which biological phenomena are developing, such as a growth of cultures of pure microorganisms or of a consortium of microorganisms (in particular microalgae), in very diverse fields such as the treatment of effluents, the production of biomass containing biomolecules of interest (i.e. biomolecules known how to be upgraded). This concept therefore encompasses in particular the reactors called fermenters.

A reactor typically includes a closed tank in which is mounted a stirring or agitation element intended to promote homogenization of the contents of the tank. Such a stirring element usually consists of a vertical shaft carrying blades or turbines whose movement, within the mass under treatment in the reactor, ensures the stirring and its homogeneity.

Various types of operating conditions may be necessary for the growth of the biological species within such a bioreactor or fermenter; autotrophic (or photo-autotrophic) growth regimes with a light supply (also referred to as photosynthesis) or mixotrophic growth regimes (with a combined carbon source and light supply) are thus in particular known. It should also be noted that the light can act on the metabolism of the cells by inducing or suppressing the production of some compounds, independently of the growth and the photosynthesis. A light supply during the culture can therefore be useful even when the microorganisms are heterotrophic.

Various configurations have already been proposed to perform such a light supply, continuously or cyclically (with cycles whose duration can vary from a few minutes to several hours), or even in the form of pulses like a flash, with a spectrum approaching that of daylight or on the contrary closely centered on a chosen wavelength.

Document WO 2014/174182 describes a reactor including a tank intended to contain a mass to be treated and provided with:
an assembly rotating about an axis intended to ensure a stirring of this mass to be treated, and
a plurality of illumination means intended to promote the treatment of this mass, the illumination means being integrated in rectangular counter-blades fastened to an inner face of the side wall of the tank, and extending in planes oriented towards the axis of rotation of the rotating assembly.

Thus, the counter-blades fastened to the inner face of the side wall of the tank have a dual function. They allow:
on the one hand, the formation of a vortex within the mass to be treated under the action of the rotating assembly, and
on the other hand, the illumination of the mass to be treated using the illumination means integrated therein.

FIG. 1 illustrates a cross-sectional view of an example of a counter-blade according to document WO 2014/174182.

The counter blade comprises:
a substantially rectangular plate 10,
a plurality of illumination means 20 mounted on an upper face of the plate 10,
electrically conductive cables 30 disposed in a central channel 40 of the plate 10, the electrically conductive cables 30 making it possible to electrically connect the illumination means 20 to an electrical energy power source (not represented),
a substantially transparent blade 50 (having the same length and width as the plate) mounted on the upper face of the plate 10 so that the illumination means 20 extend between the blade 50 and the plate 10,
a rectangular chassis 60 (having the same length and width as the plate) mounted on the rectangular blade 50, and
fastening means 70—such as bolts—to fasten together the plate 10, the blade 40 and the chassis 60.

The use of such a counter-blade allows the reactor described in document WO 2014/174182 to efficiently treat a biological mass requiring a light supply to promote its growth.

The illumination means used in document WO 2014/174182 are light-emitting diodes (or “LED”) with a 1 Watt power. The amount of light delivered by these illumination means may therefore be insufficient within the context of some treatments.

Indeed, the penetration of the light into the reactor decreases as a function of the present cell density; when the cell density is significant, it may therefore prove necessary to provide strong light so that the cells located in the center of the reactor receive light.

Furthermore, the larger the diameter of a reactor the greater the intensity of the light generated by the illumination means in order to light the cells located in the center of the reactor.

For some applications, in particular industrial applications, it would be therefore desirable to have more powerful illumination means.

However, the use of more powerful illumination means induces an increase in the consumption of electrical energy. However, within the context of industrial applications, another strong constraint consists in limiting the costs associated with the culture of photosensitive microorganisms.

An object of the present invention is to propose a reactor including a lighting device more powerful than the counter-blades described in document WO 2014/174182, while limiting the electrical energy consumption of the reactor.

### BRIEF DESCRIPTION OF THE INVENTION

To this end, the invention proposes a reactor including a tank intended to contain a mass to be treated and at least one lighting and heating device intended to promote the treatment of this mass, remarkable in that the lighting and heating device comprises:
a plate including at least one groove extending longitudinally and composed of a bottom and two side walls,
illumination means for generating a light radiation, said illumination means being disposed in the groove and being in thermal contact with the bottom of the groove, so that the heat generated by the illumination means is transmitted to the mass to be treated via the bottom of the plate.

Thus, within the context of the present invention, the heat dissipated by the illumination means is transmitted to the mass to be treated: the lighting device therefore also allows the heating of the mass contained in the reactor.

This is very advantageous, in particular in the case of the culture of Galdieria (or other strains whose culture requires both light and heat). The Galdieria strains have indeed a requirement both for brightness and for temperatures (above 35° C.) with regard to their growth conditions.

Preferred but non-limiting aspects of the assembly according to the invention are as follows:
the lighting and heating device may comprise:
at least one electrically conductive cable to electrically connect the illumination means to a remote electrical power source, the cable extending along one of the side walls of the groove,
at least one blade transparent to light radiation extending over the plate to cover the groove;
the illumination means may comprise at least one module of chip-on-board light-emitting diodes,
the material constituting the blade can be glass,
the lighting and heating device may further comprise:
at least one retaining chassis extending over the blade,
fastening means intended to cooperate with the plate, the blade and the chassis to secure the plate, the blade and the chassis;
the lighting and heating device may further comprise:
at least a first elastomeric seal between the blade and the plate, and
at least a second elastomeric seal between the blade and the chassis,

the first and second seals extending on the periphery of the blade;
each plate may comprise at least two dismountable signs joined edge to edge so as to extend in the same plane to constitute the plate;
the illumination means of a sign can be activated independently of the activation of the illumination means of the other signs;
The reactor may also comprise an agitator rotating about an axis Z to stir the mass to be treated, each lighting and heating device being fastened to an inner face of the tank and extending in a plane oriented towards the axis of the agitator to form a counter-blade that allows preventing the formation of a vortex within the mass to be treated;
the heat dissipation allows increasing the temperature of the culture medium by at least 2 degrees Celsius and thus facilitates the growth of the microorganisms.

### BRIEF DESCRIPTION OF THE DRAWINGS

Other advantages and characteristics of the assembly according to the invention will emerge more clearly from the following description of several variants, given by way of non-limiting examples, from the appended drawings in which:

FIG. 1 is a cross-sectional view of a counter-blade of the prior art as described in document WO 2014/174182;

FIGS. 2 and 3 are cross-sectional views of two embodiments of a lighting and heating device according to the invention,

FIG. 4 is a cross-sectional view of a third embodiment of the lighting and heating device,

FIG. 5 is a schematic representation of an example of a reactor including the lighting and heating device,

FIG. 6 is a curve illustrating the temperature variations in a reactor as a function of the variations in the light power of the lighting and heating device according to the invention,

FIG. 7 is a graph demonstrating the effect of heat release by lighting and heating devices according to the invention during the growth of a Galdieria sulphuraria strain.

### DETAILED DESCRIPTION OF THE INVENTION

Different examples of lighting and heating devices will now be described according to the invention allowing:
the generation of a light radiation more powerful than the counter-blades described in document WO 2014/174182, and
the generation of heat used to heat the mass to be treated.

In these different figures, the equivalent elements are designated by the same reference numeral.

As described above, even if the counter-blades according to document WO 2014/174182 allow complying with the treatment of several types of mass requiring a light supply, it may be necessary to have for some applications a more powerful illumination.

The illumination means 20 of document WO 2014/174182 consist of light-emitting diodes with a 1 Watt power.

However, the simple replacement of the 1 watt light-emitting diodes with more powerful illumination means is not an option.

Indeed, more powerful illumination means induce the generation of higher heat. This heat, if not discharged, can damage the illumination means.

However, due to the design of the counter-blade described in document WO 2014/174182, the discharge of this heat is not effective. Indeed, the central channel 40 of the plate 10 (and more specifically the air contained in this central channel 40) forms a thermal insulator preventing the discharge of the heat produced by the illumination means, so that this heat is concentrated at the illumination means and causes their premature degradation. To overcome this drawback, document WO 2014/174182 proposes the integration of heat discharge circuits within the thickness of the counter-blades.

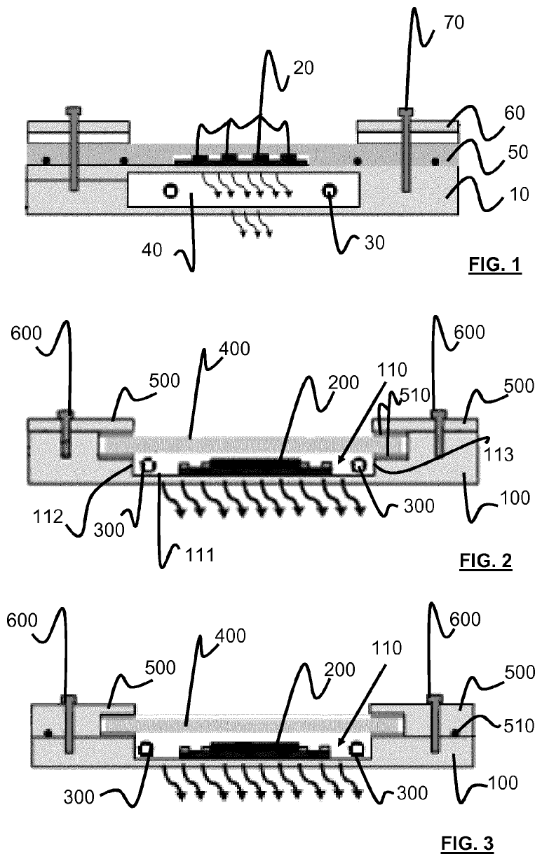

The inventors of the present invention have proposed another approach by developing a new device allowing both the lighting and the heating of a mass to be treated. Such a lighting and heating device is illustrated in FIGS. 2 to 4.

This lighting and heating device is intended to be integrated into a reactor for the treatment of a mass. It is described here with reference to the treatment of a biomass formed of microorganisms, for example microalgae; it is however understood that the following description also applies to other types of chemical or physico-chemical reactors.

The lighting and heating device will now be described in more details with reference to FIGS. 2 to 7. This lighting and heating device comprises a plate 100, illumination means 200, electrically conductive cables 300, a substantially transparent blade 400, a chassis 500 and fastening means 600.

1. Lighting and Heating Device

1.1. Plate

The plate 100 has a rectangular shape. It is preferably made of a thermally conductive material that is not very sensitive to corrosion, such as stainless steel.

The plate 100 comprises one (or several) groove(s) 110 extending longitudinally. The groove 110 is preferably rectilinear and can have any shape known to those skilled in the art in cross-section, such as a V or U shape. Each groove 110 constitutes a housing for illumination means 200 and electrically conductive cables 300. The groove 110 comprises a bottom 111 and side walls 112, 113. Advantageously, the wall constituting the bottom 111 of the groove 110 is solid to promote heat exchanges between the interior of the groove 110 and the exterior of the plate 100.

The plate 100 also comprises blind (possibly tapped) holes extending on either side of the groove 110. The blind holes allow accommodating screw pitches of fastening means 600—such as bolts—to fasten together the plate 100, the blade 400 and the chassis 500.

The plate 100 forms a support for the illumination means 200. It is advantageously dismountable with respect to the wall of the tank, therefore extractable out of the tank. Thus, an advantage is that the operations of maintaining the plate 100 and the illumination means 200 as well as the tank, are simplified. Another advantage is that the same tank can be equipped with various illumination configurations (it suffices to have several sets of plates including a different number and/or a different nature of individual illumination sources). The fact of having several sets of plates 100 including illumination configurations, different or not, also allows minimizing the interruptions in the operation during maintenance operations, since one set of plates 100 can be in maintenance while another is in service; likewise, in case of failure, the repair can be done easily, after having possibly merely replaced the plates 100 with other plates 100 provided with a similar plurality of illumination means 200.

Each plate 100 can be composed of one or several sign(s) joined edge-to-edge so as to extend in the same plane to constitute the plate 100. The fact that each plate 100 is composed of an assembly of signs makes it easier to handle the plates 100, in particular within the context of large-capacity tanks (tank of 1000 liters or more). Advantageously, the illumination means 200 carried by each sign can be activated separately. This allows activating only the illumination means 200 submerged by the mass to be treated even if this mass to be treated does not fill the entire tank.

1.2. Illumination Means

The illumination means 200 allow generating a brightness promoting the treatment of the mass to be treated. They can be of any type known to those skilled in the art.

The illumination means 200 may for example consist of a module of chip-on-board light-emitting diodes (COB LED). A COB LED module is composed of several LED chips fastened to a substrate - generally ceramic substrate. A COB LED module uses a single circuit with only two contacts to power the several diode chips installed therein. Since the COB LED module includes several chips, the light emission surface is larger, which allows obtaining a much larger light output per square centimeter. The fact that the illumination means 200 consist of modules of chip-on-board light-emitting diodes allows generating a more powerful and denser brightness.

The illumination means 200 are for example LOHAS LED Chicago, Wis. 60601.

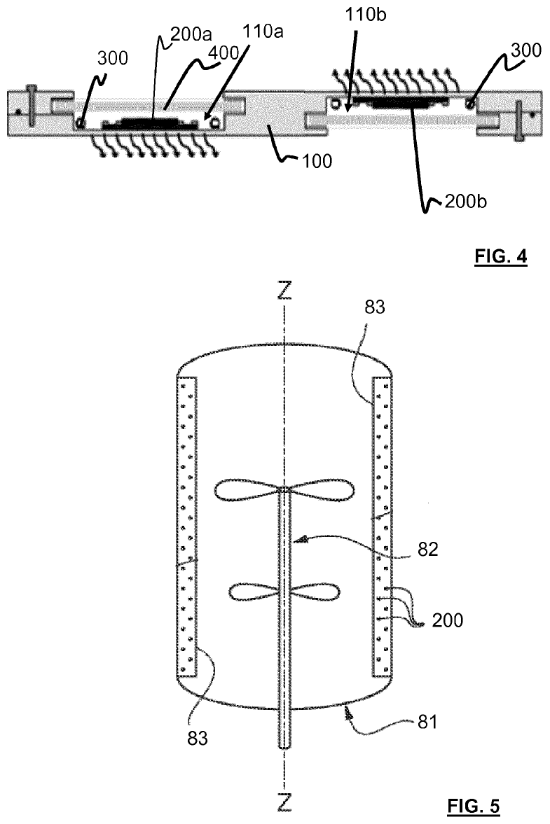

The illumination means 200 can be distributed on only one face of the plate 100 (cf. FIGS. 2 and 3) or on both faces of the plate 100. For example, with reference to FIG. 4, a variant is illustrated, in which the plate 100 comprises:
a first groove 110a extending longitudinally on one of its faces and in which first illumination means 200a are housed,
a second groove 110b extending longitudinally on the other of its faces and in which second illumination means 200b are housed,

the first and second grooves 110a , 110b being offset transversely so as to extend in parallel on either of the faces of the plate 100.

When the plate 100 comprises illumination means 200a , 200b on its two faces, the illumination means 200a on one face of the plate 100 can be distributed as a function of the distribution of the illumination means 200b on the other face, and extend transversely in the same plane or, on the contrary, be offset.

The illumination means 200 may be all identical by having the same excitation regime. As a variant, the illumination means 200 may be different. In particular, the illumination means 200 of a plate 100 may have:
separate excitation regimes (for example continuous regime for some illumination means, and flash regime at a frequency comprised between 1 and 150 kHz for other illumination means), and/or
separate emission spectra (for example in white light for some illumination means and in blue light for others), etc.

1.3. Electrically Conductive Cable

The electrically conductive cable(s) 300 allow(s) supplying the illumination means 200 with electrical energy. Advantageously, the illumination means 200 can be mounted in series or in parallel.

The cable 300 comprises electrically conductive wires intended to be connected to the power terminals of the illumination means 200 on the one hand, and of an electrical energy power source (not represented) on the other hand. The electrical wires are surrounded by an electrically insulating dielectric material. The different electrically conductive wires are contained in an outer sheath made of electrically insulating material.

The electrically conductive cable 300 extends over a side wall 112, 113 of the groove 110 of the plate 100. In the embodiment illustrated in FIG. 2, the plate comprises two electrically conductive cables 300, each cable 300 extending along a respective side wall 112, 113 of the groove. This allows avoiding the presence of obstacle to light radiation in line with the illumination means 200.

1.4. Transparent Blade

The blade 400 allows covering the groove 110 forming a housing for the illumination means 200. When the lighting and heating device comprises illumination means 200 distributed on both faces of the plate 100, the device comprises first and second blades 400 extending respectively on the first and second faces of the plate 100, each blade making it possible to cover a respective housing of the lighting and heating device.

Each blade 400 is made of a material transparent to light radiation, such as a polysulfone resin. However, the material constituting each blade 400 is preferably glass. The use of glass allows ensuring better sealing of the housing. The glass also has the advantage of being more transparent than the resin, and of better resisting:
the temperature and pressure constraints during sterilizations, and
the base (NaOH) or acid (H2SO4 . . . ) based treatments used during reactor cleaning cycles.

Each blade 400 may comprise through-openings extending in line with the blind holes of the plate 100 to allow the passage of the fastening means 600.

1.5. Chassis and Fastening Means

The chassis 500 consists of a retaining profile of the same surface area as the plate 100. In the embodiment illustrated in FIG. 2, the chassis consists of a substantially planar frame, the plate comprising a recess at the periphery of the groove to allow accommodating the side edges of the blade 400. In the embodiment illustrated in FIG. 3, the chassis 500 differs from the embodiment illustrated in FIG. 2 in that it is the one that comprises a recess on its internal edge to allow accommodating the sides of the blade 400.

The chassis 500 is made of a material that is not very sensitive to corrosion, such as stainless steel.

It is intended to be positioned above the blade 400 and to be bolted on the plate 100 to allow fastening the blade 400. Elastomeric seals 510 (EPDM, Viton . . . ) can be positioned at the periphery of the blade 400:
on the face of the blade 400 intended to come into contact with the plate 100 so as to surround the groove 110 and ensure the sealing between the blade 400 and the plate 100, and
on the face of the blade 400 intended to come into contact with the chassis 500 so as to ensure the sealing between the blade 400 and the chassis 500.

Advantageously, the plate 100 and the chassis 500 may comprise ribs (not represented) to ensure a proper anchoring of seals.

Furthermore, the chassis 500 may comprise through-apertures intended to be positioned in line with the blind holes of the plate 100 and through-openings of the blade 400 for the passage of the fastening means 600.

These fastening means 600 can consist of bolts that allow securing the plate 100, the blade(s) 400 and the chassis 500. The fastening means 600 may complementarily comprise a bonding material, or any other fastening means known to those skilled in the art.

2. Example of a Reactor Integrating the Lighting and Heating Device

2.1. Illustration of the Heat Generation

With reference to FIG. 5, an example of a reactor implementing the present invention has been illustrated.

The reactor mainly includes:
a tank 81 intended to contain a mass to be treated,
a rotating assembly 82 about an axis Z-Z intended to ensure a stirring of this mass of microorganisms, and
a plurality of lighting and heating devices 83 intended to promote the growth of this mass of microorganisms.

The tank includes an inner wall to which are fastened lighting and heating devices 83 whose planes are oriented towards the axis of the rotating assembly 82 and parallel thereto so as to prevent the formation of a vortex within the mass of microorganisms under the action of the rotating assembly 82. The lighting and heating devices 83 thus constitute counter-blades.

Such a reactor may include, depending on its application, other elements (not represented) such as, in particular, a channel for the input of a product to be treated or degraded by means of the biomass, or a channel for the supply with reagents or nutrients such as sugars for the proliferation of the biomass, a channel for the intake and exit of air or gas, or a channel for the withdrawal of microorganisms.

Upon activation of each lighting and heating device 83, the illumination means 200 generate light radiation intended to promote the growth of the mass of microorganisms or induce the production of compounds of interest: Indeed, the light can act on the cell as a signal to modify the metabolism independently of the photosynthesis. Along with the generation of light, the illumination means 200 produce heat. This heat is dissipated via the bottom 111 of the groove 110, the latter being in thermal contact with the illumination means 200.

Thus, the characteristics of the lighting and heating device allow better dissipation in the culture medium of the heat generated by the illumination means 200 and an increase by a few degrees of the culture medium or the fermentation must.

The heat release of two lighting and heating devices 83 was measured in a fermenter containing 95 liters of water, under agitation at 300 rpm, without air flow rate to limit the evaporation during the experiment and keep a constant volume in the tank during the experiment. The temperature regulation of the tank by double jacket is not activated, the temperature of the part is controlled and constant near 22° C. The temperature is monitored by an inner probe with a reading every two minutes.

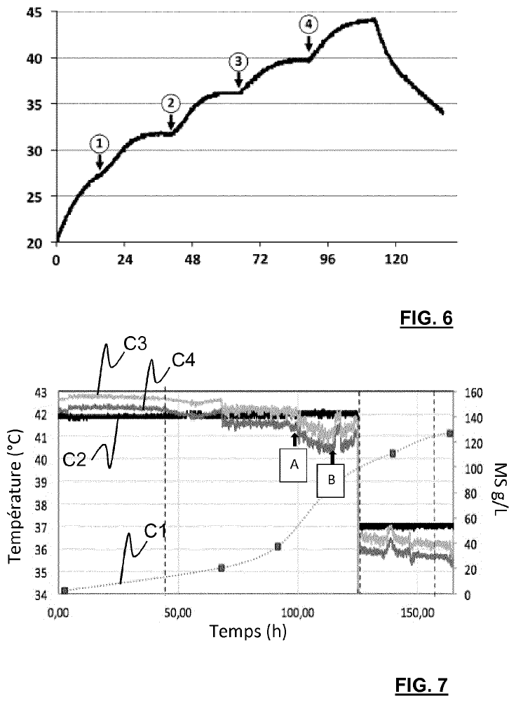

The results of this experiment are illustrated in FIG. 6. After the first 15 hours, the temperature rise 1 from 20° C. to 27° C. is linked to the agitation of the mixing axis.

The lighting and heating devices are then activated (illumination means generating a light radiation) and the power is increased in increments of 25%, thus delivering 48 watts, 96 watts, 144 watts and 192 watts respectively, which corresponds to 0.48 w/L; 0.96 w/L; 1.44 w/L and 1.92 w/L.

Each power level causes an increase in temperature 2, 3, 4 of about 5° C. in less than 24 hours. This results in an increase of the water temperature of more than 20° C., and a final temperature of 44° C. for a power of 1.92 w/L.

When the lighting and heating devices are deactivated (illumination means no longer generating light radiation), a drop in water temperature is observed almost immediately.

In addition to the fact that this system allows heat dissipation from the counter-blades towards the culture medium in order to maintain an acceptable operation temperature for the illumination means, it allows saving energy when the cultures of microorganisms such as algae also require heating the culture medium.

As an example, strains like Galderia sulphuraria whose optimum growth is between 42° C. and 45° C. can be mentioned. The increase in temperature of the culture medium is an advantage in this case.

The graph in FIG. 7 illustrates the effect of the release of heat from the lighting and heating device during the growth of Galderia sulphuraria in a bioreactor including a double-wall in which a heat transfer fluid circulates (here water) allowing to maintain the culture medium of Galderia sulphuraria at a desired temperature.

This graph comprises:
A first curve C1 representative of the growth of the Galderia sulphuraria strain expressed in g of dry matter per liter of culture,
A second curve C2 corresponding to the recording of the temperature of the culture medium inside the reactor, the temperature of the culture medium being kept constant at 42° C. until time 125 h, then the temperature of the culture medium being deliberately lowered to 37° C. thereafter until the end of the culture,
A third curve C3 corresponding to the recording over time of the input temperature of the heat transfer fluid used to regulate the temperature of the culture medium, and
A fourth curve C4 illustrating the output temperature of the heat transfer fluid.

At 100 hours of fermentation (point A), the lighting and heating devices are activated. The temperature of the culture inside the tank does not fluctuate and remains stable at 42° C. while the input and output temperatures of the heat transfer fluid contained in the double-jacket decrease by several degrees in a few hours. These recordings clearly reflect the effect of diffusion of the heat generated by the illumination means towards the culture medium via the metal structure of the lighting and heating device.

Reversibly, when the lighting and heating devices are deactivated (point B at about 115 h), the input and output temperatures of the heat transfer fluid increase, reflecting the necessity to heat this heat transfer fluid to keep the temperature of the culture medium constant.

At 125 hours, the temperature of the culture medium was deliberately lowered to 37° C., and the lighting and heating devices were reactivated. Similarly to what has been observed at 42° C., a gradual decrease in the temperature of the heat transfer fluid contained in the double-jacket is observed. The light power used during this experiment is of 2 w/L, and an ambient temperature is of 22° C. It is therefore conceivable to be able to widely increase the power of the illumination means of the lighting and heating devices, to 4 w/l or even up to 6 w/l for example without this involving a cooling of the heat transfer fluid contained in the double-jacket.

FIG. 7 demonstrates that without lighting and heating device, the heat transfer fluid contained in the double-wall of the reactor (and which allows regulating the temperature of the culture medium) must be continuously heated to maintain the temperature of the culture medium at a setpoint value.

3. Conclusions

The lighting and heating device described above allows increasing the amount of light delivered to a mass to be treated. This is valid for cultures in mixotrophy, in autotrophy on photosynthetic organisms but also for cultures in mixotrophy predominantly heterotroph where the light is not important for the photosynthetic activity but for example for the induction of molecules of interest such as pigments (document WO2017050917), and/or oil.

This device also allows increasing the temperature of the culture medium.

The reader will have understood that several modifications can be made to the invention described above without physically departing from the new teachings and advantages described here.

For example, in the description above, the lighting and heating device was integrated into a reactor including a rotating assembly intended to ensure a stirring of this mass of microorganisms. It is obvious to those skilled in the art that the lighting and heating device described above could be integrated into a reactor devoid of rotating assembly.

Consequently, all modifications of this type are intended to be incorporated within the scope of the appended claims.

What is claimed is:

1. A reactor including a tank intended to contain a mass to be treated and at least one lighting and heating device intended to promote the treatment of this mass, wherein the lighting and heating device comprises:
a plate including at least one groove extending longitudinally and composed of a bottom and two side walls,
illumination means for generating a light radiation, said illumination means being disposed in the groove and being in thermal contact with the bottom of the groove, so that the heat generated by the illumination means is transmitted to the mass to be treated via the bottom of the plate.

2. The reactor according to claim 1, wherein the lighting and heating device comprises:
at least one electrically conductive cable to electrically connect the illumination means to a remote electrical power source, the cable extending along one of the side walls of the groove,
at least one blade transparent to light radiation extending over the plate to cover the groove.

3. The reactor according to claim 1, wherein the illumination means comprise at least one module of chip-on-board light-emitting diodes.

4. The reactor according to claim 2, wherein the material constituting the blade is glass.

5. The reactor according to claim 1, wherein each plate comprises at least two dismountable signs joined edge to edge so as to extend in the same plane to constitute the plate.

6. The reactor according to claim 5, wherein the illumination means of a sign can be activated independently of the activation of the illumination means of the other signs.

7. The reactor according to claim 1, further comprising an agitator rotating about an axis Z to stir the mass to be treated, each lighting and heating device being fastened to an inner face of the tank and extending in a plane oriented towards the axis of the agitator to form a counter-blade that allows preventing the formation of a vortex within the mass to be treated.

8. The reactor according to claim 1, wherein the lighting and heating device is arranged so that the heat dissipation allows increasing the temperature of the culture medium by at least 2 degrees Celsius and thus facilitates the growth of the microorganisms.
